(19) United States
(12) Patent Application Publication
(10) Pub. No.: US 2003/0215397 A1
(43) Pub. Date: Nov. 20, 2003

(54) Method of treatment of skin diseases related to microbial antigens in the skin

(76) Inventor: Patricia W. Noah, Germantown, TN (US)

Correspondence Address: Barry G. Magidoff, Greenberg Traurig, LLP, 885 Third Avenue, New York, NY, 10022, US

(21) Appl. No.: 10/436,311

(22) Filed: May 12, 2003

Related U.S. Application Data
(60) Provisional application No. 60/380,698, filed on May 14, 2002.

(51) Int. Cl. A61L 9/04; A61K 31/23

(52) U.S. Cl. 424/44; 514/552

(57) ABSTRACT

A method of treating isomorphic diseases of the skin of mammals, to reduce the lesions or roughing of the skin surface resulting from such diseases, the method comprising topically applying to the skin of a mammal suffering from the skin disease several doses of an effective amount of a liquid composition containing a clinically effective concentration of a fatty acid ester of an alcohol, the ester comprising a total of from about 10 to about 35 carbon atoms, and a carrier liquid, the carrier liquid comprising water and a volatile parafinnic alcohol having up to four (4) carbon atoms, the proportion of water in the carrier liquid being from 0 to about 75% by volume, and further comprising a surfactant in the amount of up to about 0.4% by wt. of the total composition, the carrier liquid being present in an amount of from about 0 to about 60% by vol. of the total liquid composition, the ester being dispersed with any carrier liquid present. Examples of suitable active esters are the palmitates, laurates, and myristates.

This invention relates to a treatment for psoriasis and other skin disorders which are characterized by redness of the skin, itching, flaking, scaling and plaque-type growth. More particularly, the present invention relates to formulations for treating such disorders, based upon a newly discovered microbial basis for such diseases, or at least for the symptoms resulting from such diseases. These formulations comprise an ester of a fatty acid and fatty alcohol which serve to remove microbial material from the lesional skin of psoriasis patients and/or from the epidermal basement membranes of individuals having normal-looking skin, but who suffer from the disease. The esters may be provided in a pharmaceutically acceptable carrier. This invention also provides a method of treating individuals having the undesirable skin disorder, but without the resulting lesions or plaques, by solubilizing deposited microbial agent antigen materials located on or in the skin, and thus cleansing the skin of the antigens. These antigen materials are deposited at the skin basement membrane by way of capillary circulation.

### BACKGROUND INFORMATION

Chronic skin disorders have been disruptive or dangerous ailments affecting a variety of mammals, and which are characterized by redness of the skin, itching, flaking, scaling, plaque-type growth and the loss of hair or fur. It has long been known to treat these ailments with a variety of materials. Specifically it has been known to utilize as an active ingredient, either alone or in combination with other known active materials, isopropyl myristate for the treatment of psoriasis, and other skin diseases. The other known active ingredients include compounds such as corticosteroids, e.g. hydrocortisone or clobetasol propionate, as well as other active materials such as zinc pyrithione, coal tar, salicylic acid, selenium sulfide. It is has also been known to use the isopropyl myristate as an emollient, together with other active ingredients, as part of a typical treatment. In addition, it was recognized that other fatty acid esters could be utilized as emollients, including isopropyl palmitate, octylpalmitate, isononanoate and isocetyl stearate. These esters were recognized primarily as being useful for enhancing the absorption into the skin of therapeutically active ingredients. The use of isopropyl myristate as an active ingredient is shown for example in U.S. Pat. Nos. 5,886,038 and 5,990,100, to the present applicant and others, for application to the areas of the skin having psoriatic lesions.

### SUMMARY OF THE INVENTION

It has now been discovered by the present inventor, working together with others, that Isopropyl myristate acts in a completely unexpected way having application to a variety of skin disorders other than psoriasis, which disorders all include the so-called Koebner phenomenon. The Koebner phenomenon involves an isomorphic response, i.e., wherein patients with the active skin disease may have normal-looking skin until such skin is traumatized, and the disease then becomes visibly active at the injured sites.

In accordance with the preferred process of this invention, the normal-looking, as well as the lesional, skin of mammals suffering from a Koebner type skin disease is cleansed of microbial antigens, using the fatty acid esters of the present invention. Such a cleansing will solubilize and remove from any lesional epidermis, and from the epidermal basement membranes of nonlesional skin, substantially all microbial antigens. The examples of skin diseases which do exhibit the Koebner phenomenon include, among others, the following:

Psoriasis

Lichen Planus

Erythema Multiforme

Drug rashes

Sarcoidosis

Granuloma annulare

Lupus erythematosus

Pemphigoid

Pemphigus

Atopic eczema

Seborrheic dermatitis

Perioral dermatitis

The active ingredients in accordance with the present invention can be applied utilizing a spray formulation or an aerosol, wherein the active fatty ester is dissolved in a low molecular weight alcohol, such as ethyl alcohol, or isopropyl alcohol, or a cream, or in any liquid or ointment form. The antigens which create the problem on the skin are believed to be phospoglycolipids.

The active esters are preferably present in amounts of from 20% by wt. to about 100 percent by weight, depending upon the requirements of solubility and viscosity, and most preferably in an amount of at least 35% by wt., and even more preferably at least about 40% by wt., and most preferably not more than about 95% by wt. It is generally known that these materials are soluble in the lower alcohols alone or mixed with water. Generally, when presented as a flowable liquid, the esters are disbursed in a lower molecular weight alcohol, or alcohol and water, preferably present in a volume ratio of from about 5:1 to about 20:1 alcohol:water.

The esters which provide the active ingredients in accordance with the present invention can be defined by the following general formula:

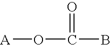

wherein, A and B are each one of an alkyl or alkenyl group, straight or branched chain, having a total of between about 13 and about 35 carbon atoms, preferably from about 13 to about 19 carbon atoms. Most preferably, one of A and B has from one (1) to three (3) carbon atoms, and optimally “A-O”—” is the residue of an alcohol having 2 or 3 carbon atoms.

More particularly, the preferred saturated esters of this invention can be described by the following structural formula:

$$[ {( {{CH}_{3} ( {CH}_{2} )}_{n} )}_{w} — {{C}_{{( {{CH}_{3} ( {CH}_{2} )}_{m} )}_{v}}}^{{H}_{z}} —o— {C}^{o} {— ( {CH}_{2} )}_{x} {—CH}_{3} ]$$

In the above structural formula, the sum of the values w+z+v total 3; where, w=0, 1 or 2 and preferably 1; v=0 or 1; z=0, 1, 2 or 3, preferably 1 or 2; the values of m and n is 0 to about 8, preferably 0, 1 or 2; and the sum of the products of (w(n+1)) plus (v(m+1)) is “S”, and “S” equals from 0 to about 21; x equals zero or an integer from 1 to about 20 carbon atoms and preferably from 10 to about 16 carbon atoms, and the sum of (S+x+3) equals from about 14 to about 32, and preferably from about 16 to about 24.

Preferred such compounds are the derivatives of lower molecular weight fatty alcohols, having 1 to 4 carbon atoms, and fatty acids having 10 to 18 carbon atoms, including the carboxyl group.

Examples of such useful compounds, within the above the general categories, include cetyl 2-ethyl-hexanoate: isopropyl myristate; isopropyl palmitate; 2-ethylhexyl palmitate; 2-octyldodecyl myristate; butyl stearate; 2-ethylhexyl stearate and isopropyl laurate. These esters which have been accepted for cosmetic and/or pharmaceutical purposes include cetyl 2-ethyl hexanote; isopropyl palmitate; 2-ethylhexyl palmitate; 2-octyldodecyl myristate; butyl stearate; 2-ethylhexyl stearate; octyl isononanoate; and isocetyl stearate. These compounds are not only effective in providing the antigen solvent effect, but also have long been used in topical applications, whether for cosmetic purposes or as carriers in topical pharmaceuticals. The goal is to incorporate a material into the lipid bilayer of the basement membrane and render the microbial antigens and toxins into a more liquid state, detaching them from the bilayer and thus expediting the movement of the antigens to the skin surface, from where they are readily washed away.

A useful composition for the ready topical application of these active compounds comprises the following: a lower molecular weight alcohol including ethyl alcohol (preferably denatured) or isopropyl alcohol, preferably mixed with a minor proportion of water. Such a carrier is effective in a spray or aerosol application. Other ingredients in such a useful composition include a surfactant/emulsifier, such as sodium lauryl sulphate, Tween 80, or Polysorbate 80, and other known surfactants useful in medicinal or veterinary treatments. Generally, the greater the amount of the alcohol carrier, the lower the requirement for a surfactant. As the quantity of water increases the need for a surfactant or emulsifier also increases. Generally, the ratio of alcohol to water is in the range of approximately 1:12 to about 40:1, and preferably in the range of from about 2:1 to about 20:1. It is generally useful to balance the proportion of alcohol in the composition, to avoid the tendency for alcohol to cause irritation and excessively dry the skin, especially when being applied to damaged skin surfaces, and the desire to avoid the oily feel when higher proportions of water are used. For this reason, it could be useful to add emollients or humectants or other customary additives used in such topical treatments. The esters of the present invention provide their cleansing effect by loosening or releasing the antigens from the membrane bilayer, which serves to accelerate the migration of the undesirable antigens to the skin surface and away from the skin basement membrane. This tends to swiftly reduce the quantity of the undesirable antigens on the membrane. The composition is preferably applied repeatedly over a period of time, such as at least during about 1 week and preferably during about two weeks.

As the proportion of water in the carrier liquid increases, emulsifiers become more useful to disperse or solubilize the fatty acid ester, and/or other anti-psoriatic agent. Suitable emulsifiers are known in the art, especially sodium lauryl sulphate and Polysorbate 80 (polyoxyethylene (20) sorbitan monooleate) are useful to help solubilize or emulsify or disperse the esters in the carrier liquid, especially when the proportion of water is high. Most of the esters, such as ethyl myristate, tend to be very soluble or miscible in ethanol, or isopropyl alcohol, so that when the carrier liquid is predominantly such an alcohol, the emulsifiers may be unnecessary and may be omitted. Other emulsifiers which could by useful in the compositions of the present invention, include the various Tween compounds, the various ethanolamines, such as triethanolamine, the various SPAN compounds, dioctyl sodium sulfosuccinate, Celouacrogols, Tensides, etc. These emulsifiers may be used singly or in combination with other surfactants. The emulsifiers preferably form less than 10 weight percent of the formulation, most preferably less than about 5%, and most preferably not more than about 3% by weight of the emulsifier is present. Optimally, when high concentrations of a lower alcohol, ethanol or isopropanol, is present, far lower levels of, or even zero, surfactant will be satisfactory.

For most of the esters useful in this invention, the vehicle is preferably a combination of a relatively small amount of water with the alcohol, such as not more than about 5% water. The present invention is intended to act as a cleansing process, and thus the fatty acid ester should be applied and left on the skin, during repeated applications. The ester acts to loosen any antigens on the lesion surface, and it will also diffuse or permeate through any unbroken skin to the basement membrane, and loosen any antigens on the membrane. This will result in the elimination of the antigens during the normal sloughing action of the skin. Useful formulations include a liquid, such as a shampoo, or liquid soap, or a solid soap which can be applied and readily solubilized to act on the skin by applying a suitable liquid, such as water, or an alcohol/water mixture, or even a relatively viscous lotion or a cream or ointment.

In compositions containing alcohol, where it is desired to avoid the drying, and possible irritant effect of alcohol, higher values of water, but preferably not more than about 25% water to 75% alcohol can be used. The higher proportions, especially a major proportion of water-to-alcohol, require larger amounts of surfactants and may create an oily or greasy feel to the product. Emollients, humectants, and other customary additives may be added, to overcome the drying effect of the alcohol in the carrier, in place of increasing the proportion of water. Sufficient water, on the other hand should be used to reduce the drying effect of the alcohol, and from another point of view, to reduce the flammability of the vehicle. The greater the proportion of water, the more the flash point will be increased, making the product less hazardous to make and use.

Ethanol, preferably 96% alcohol, is the preferred carrier alcohol. Less preferably isopropyl alcohol can be used or other lower alcohols, such as methanol or a butyl alcohol, or combinations thereof, preferably none of the alcohols has more than four (4) carbon atoms, and alkyl alcohols are preferred over alkenyl alcohols.

The active ester compound of this invention is preferably at least 30 weight percent of the composition, and most preferably at least 40%. The higher proportions are most preferred, as long as a suitably fluid composition is obtained. A preferred pharmaceutical composition according to the invention is, by weight percent, 40% fatty acid ester, such as isopropyl palmitate, 0.1% sodium lauryl sulphate, 1.5% Polysorbate 80, 3.4% water, and about 55% ethanol.

The following examples illustrate the preferred aspects of the present invention based upon the finding that the fatty acid esters useful in the present invention are effective to cleanse away microbial antigen products that are deposited in or on the skin of mammals suffering from skin lesions, such as are formed by diseases such as psoriasis.

In general, it has been found that a mammal suffering from psoriasis, whose past symptomatic skin lesions significantly worsened following streptococcal infection, can be effectively treated in accordance with this invention. Anti-body levels of many such patients, to streptococcal exoenzymes, deoxyribonuclease B, hyaluronidase, streptolysin O, and Streptozyme (a commercial product made from streptococci and containing Dnase-B as one of its constituents., a registered trademark of Carter-Wallace, Inc. and available from Wampole Labs, of Cranbury, N.J.), when measured will be found to be elevated prior to the treatment in accordance with this invention. The presence of these antigens are tested for by using a primary rabbit polyclonal antibody to, e.g., Streptozyme® with a linked streptavidin biotin alkaline phosphatase (“LSAB”) amplification stain, available from Dako, of Carpinteria, Calif. A prominent red stain on the biopsied skin indicates the presence of the antigens.

A 34-year old man, suffering from severe psoriasis came for treatment. His psoriasis had become severe at a time about 20 years earlier when he had recurring sore throats and tonsilitis. His sore throats had ceased, but his psoriasis persisted. A blood sample was taken to measure his antibody level to Dnase-B, a streptococcal exoenzyme; It was determined that he was a streptococcal carrier. A 4 mm punch biopsy was taken at the same time from a psoriatic plaque on his posterior thigh. The biopsy was first exposed to an antibody to Streptozyme®, and stained using the LSAB test. A positive, red stain, showed the presence of the streptococcal toxin (Dnase-B) in the skin.

The patient was provided with a pump spray bottle containing 40% by weight of isopropyl myristate in an alcohol carrier, and instructed to spray the liquid on one particular lesion twice a day for two weeks. At the end of the two weeks he returned, and the lesion spot he had sprayed during that period was now substantially clear of the disease. That site was biopsied and given the LSAB test, as before. The stain was negative, indicating the cleansing of the streptococcal antigen from the skin that had been treated with the 40% isopropyl myristate spray, and it was accompanied by a clearing of the lesion.

The following ester compositions are tested by the above procedure, and will show the effectiveness of other exemplary fatty acid ester compounds in the formulations of this invention:

| Ingredients | Parts By Wt. |
|---|---|
| Cetyl 2-ethyl hexanoate | 40 |
| Isopropyl palmitate | 40 |
| Cetyl palmitate | 40 |
| Cetyl stearate | 40 |
| Isopropyl stearate | 40 |
| 2 ethyl hexyl palmitate | 40 |
| Octyldodecyl myristate | 40 |
| Butyl stearate | 40 |
| 2 ethyl hexyl stearate | 40 |
| Isocetyl stearate | 40 |
| Octyl isononanoate | 40 |
| Butyl undecylenate | 40 |
| Ethyl laurate | 40 |
| Myristyl laurate | 40 |
| Methyl myristate | 40 |
| Ethyl myristate | 40 |
| Methyl laurate | 40 |
| Ethyl Palmitate | 40 |
| Isopropyl laurate | 40 |

Each of the above fatty acid esters is tested in the following sprayable carrier liquid:

| Ingredients | Parts by Wt. |
|---|---|
| Sodium Lauryl Sulphate | 0.1 |
| Polysorbate 80 | 1.5 |
| Water | 5 |
| Ethanol | 65 |

The composition in each case is liberally applied and the patient otherwise is permitted to carry out the usual hygiene, showering or bathing. It is believed that the active ester ingredient dissolves or releases the antigens from the skin surface and/or from the skin basement membrane, and the normal sloughing action of the skin results in the discharge and cleansing of the antigens from the skin, over a period of time. It is believed that the higher the concentration of the active ester compound in the composition the more effective the action will be. Those esters that have a suitable liquid property, can be applied in a substantially pure state.

Each patient will be substantially free of antigen material at the treated sites, following approximately two weeks of treatment. The appearance of the skin lesions also markedly improves, at those sites. The results of the above Examples are surprising and unexpected.

Each of the above compositions resulted in the substantial disappearance of any indication of antigenic materials in the skin or on the basement membrane, as well as distinct improvement in the appearance of any lesions. Although the preferred embodiments have been described, it is understood that various modifications, and other esters within the broad range defined, may be used without departing from the scope of the invention as disclosed herein.

The following embodiments are claimed.

1. A method of treating isomorphic diseases of the skin of mammals, which diseases are manifested by lesions or roughing of the skin surface, the method comprising topically applying to the skin of a mammal suffering from the skin disease several doses of an effective amount of a liquid composition containing a clinically effective concentration of a fatty acid ester of an alcohol, the ester comprising a total of from about 10 to about 35 carbon atoms, and a carrier liquid, the carrier liquid comprising water and a volatile parafinnic alcohol having up to four (4) carbon atoms, the proportion of water in the carrier liquid being from 0 to about 75% by volume, and further comprising a surfactant in the amount of up to about 0.4% by wt. of the total composition, the carrier liquid being present in an amount of from about 0 to about 60% by vol. Of the total liquid composition, the ester being dispersed with any carrier liquid present.

2. The method according to claim 1 wherein the liquid composition is applied as a spray.

3. The method according to claim 1 comprising in addition a compound selective from the group consisting of soaps and detergents.

4. The method according to claim 1 wherein the composition is applied to the surface of the skin having lesions and to skin apparently free of such disease symptoms, repeatedly at predetermined intervals, so that the symptoms are relieved.

5. The method according to claim 1 wherein the fatty acid ester is the ester of an aliphatic alcohol having from one (1) up to about 14 carbon atoms and a fatty acid having from about six (6) up to about 20 carbon atoms, wherein the combined total number of carbon atoms is in the range of from at least about 12 up to about 35.

6. The method according to claim 1 wherein the aliphatic alcohol is selected from the group consisting of alkyl alcohols and alkenyl alcohols, and the fatty acid is selected from the group consisting of alkyl fatty acids and alkenyl fatty acids.
